(19) United States
(12) Patent Application Publication
(10) Pub. No.: US 2023/0151336 A1
(43) Pub. Date: May 18, 2023

(54) STEM CELL DERIVED SINGLE-ROSETTE BRAIN ORGANOIDS AND RELATED USES THEROF

(71) Applicant: The Regents of the University of Michigan, Ann Arbor, MI (US)

(72) Inventors: Jack Parent, Ann Arbor, MI (US); Andrew Tidball, Ann Arbor, MI (US)

(21) Appl. No.: 17/920,599

(22) PCT Filed: Apr. 22, 2021
(86) PCT No.: PCT/US2021/028610

§ 371 Date: Oct. 21, 2022

Related U.S. Application Data
(60) Provisional application No. 63/013,608, filed on Apr. 22, 2020.

(51) Int. Cl. C12N 5/071; G01N 33/50

(52) U.S. Cl. CPC C12N 5/0697; G01N 33/5082; C12N 2506/45; C12N 2501/15; C12N 2501/155; C12N 2503/04; C12N 2501/415; C12N 2501/41; C12N 2533/90; C12N 2513/00

(57) ABSTRACT

The invention disclosed herein generally relates to methods and systems for converting stem cells into specific tissue(s) or organ(s) through directed differentiation. In particular, the invention disclosed herein relates to methods and systems for promoting human self-organizing single-rosette spheroids (SOSRS), a type of brain organoid, comprising neuroepithelium having either a dorsal cell fate or a ventral cell fate formation from pluripotent stem cells.

### CROSS-REFERENCE TO RELATED APPLICATION

The present application claims priority to U.S. Provisional Patent Application Ser. No. 63/013,608 filed Apr. 22, 2020, which is hereby incorporated by reference in its entirety.

### FIELD OF THE INVENTION

The invention disclosed herein generally relates to methods and systems for converting stem cells into specific tissue(s) or organ(s) through directed differentiation. In particular, the invention disclosed herein relates to methods and systems for promoting human self-organizing single-rosette spheroids (SOSRS), a type of brain organoid, comprising neuroepithelium having either a dorsal cell fate or a ventral cell fate formation from pluripotent stem cells.

### INTRODUCTION

Many individuals will be affected by neurological disease, neurodevelopmental disease, and/or neuropsychiatric disease (e.g., autism, anxiety, mood disorders, epilepsy, neurodegenerative disease). The cost of brain disease is estimated to be hundreds of billions of dollars per year. Neuroscience has typically relied on the experimental manipulation of living brains or tissue samples, but scientific progress has been limited by a number of factors. For ethical and technical reasons, most invasive techniques are impossible to use on humans. Experiments in animals are expensive and results obtained in animals must be verified in long and expensive human clinical trials.

Improved experimental models of the human brain are urgently required to understand disease mechanisms and test potential therapeutics.

The present invention addresses this need.

### SUMMARY

The field of brain organoid technology began in the lab of Yoshiki Sasai in 2008 when they generated 3-dimensional cortical structures from mouse embryonic stem cells (ESCs) (see, Eiraku, M. et al. Cell Stem Cell 3, 519-532 (2008)). Five years later, they developed a technique for generating the same structures (SFEBqs) from human ESCs (see, Kadoshima, T. et al. Proceedings of the National Academy of Sciences 110, 20284-20289 (2013)). This method uses WNT and TGFβ inhibition to pattern an embryoid body, a sphere of human pluripotent stem cells, into neuroepithelial tissue. The neuroepithelial tissue then self-organizes to form cortical rosette structures. These organizing centers are radially organized around a central lumen and are in vitro correlates of the neural tube that forms in embryonic development. While embryos only have a single neural tube, brain organoids contain many of these organizing centers. Other labs developed similar techniques to generate what began to be termed brain organoids, but in each case multiple rosettes form (see, Lancaster, M. A. et al. Nature 501, 373 (2013); Paşca, A. M. et al. Nature Methods 12, 671 (2015)). The variability of rosette formation is likely the cause of an overall problem of structural heterogeneity in the brain organoid field (see, Quadrato, G. & Arlotta, P. Current opinion in cell biology 49, 47-52 (2017)). For this reason, many groups have used single-cell RNA-sequencing to investigate development and disease as a non-structural read-out (see, Quadrato, G. et al. Nature 545, 48 (2017); Camp, J. G. et al. Proceedings of the National Academy of Sciences 112, 15672-15677 (2015)). Most disease phenotypes observed to-date in brain organoids cause a severe size difference such as microcephaly or macrocephaly (see, Lancaster, M. A. et al. Nature 501, 373 (2013); Li, Y. et al. Cell stem cell 20, 385-396. e383 (2017); Qian, X. et al. Cell 165, 1238-1254 (2016); Iefremova, V. et al. Cell reports 19, 50-59 (2017)).

The structural heterogeneity and multiple organizing centers (rosettes) in brain organoids do not occur in other organoid systems. For example, intestinal (see, Spence, J. R. et al. Nature 470, 105 (2011)) and pancreatic (see, Huang, L. et al. Nature medicine 21, 1364 (2015)) organoids have a single lumen structure around which they organize. One main difference between these methods is that non-CNS organoids typically begin with patterning a 2-dimensional (2D) culture of pluripotent stem cells followed by 3-dimensional induction of a thick sheet of extracellular matrix (ECM) proteins. These techniques are used for intestinal, pancreatic (see, Huang, L. et al. Nature medicine 21, 1364 (2015)), liver, and kidney organoids. Additionally, normal neural development begins with patterning of the neural plate, a 2D structure, followed by neurulation to form the 3D neural tube. Accordingly, experiments were conducted by the inventors of the technology described herein based on a hypothesis that following standard techniques for generating 2D cortical neuroepithelium followed by 3D structure formation on ECM would result in brain organoids with a single organizing center (rosette).

Based on these insights, experiments conducted during the course of developing embodiments for the present invention transferred small clumps of dual-SMAD differentiated neuroepithelium onto Geltrex, an ECM-like product and found rapid self-organization of single lumen structures expressing apical markers (e.g., Self-Organizing Single Rosette Spheroids or SOSRS). Over time in culture, the SOSRS were shown to develop normal inside-out lamination, markers of deep and superficial cortical neurons, and astrocytes. The consistent size and architecture of the SOSRS allowed for refined measurements by both immunostaining and bright-field imaging.

Specifically, such experiments resulted in the generation of SOSRS that generate excitatory cortical-like neurons. A key technical difference was starting with a 2-dimensional monolayer of pluripotent stem cells (PSCs) that were patterned with small molecules into neuroepithelium rather than patterning a 3-dimensional sphere of human PSCs termed an embryoid body. To pattern such experiments utilized a cocktail of four small molecule inhibitors (DMH1, XAV939, SB431542, and cyclopamine) in N2B27 culture medium. After 4 days of patterning, this monolayer was cut into uniformly sized pieces using the StemPRO EZPASSAGE tool (Thermo Fisher). The pieces were then passaged onto solidified, undiluted Geltrex (Thermo Fisher), a gelatinous basement membrane matrix. Within 4 days, the vast majority of monolayer fragments formed consistently sized spheres with a single central lumen. At this time, the cells in these spheres labeled with markers for neuroepithelium and radial glial cells, the stem cells of the developing cortex. The SOSRS were shown to have a consistent diameter and rapid growth, producing neurons from multiple cortical layers. It was also shown that replacement of cyclopamine with the sonic hedgehog pathway activator, SAG, generated SOSRS expressing markers of ventral forebrain. Over time, these SOSRS were shown to generate a large number of GABAergic (inhibitory) interneurons.

Accordingly, the invention disclosed herein generally relates to methods and systems for converting stem cells into specific tissue(s) or organ(s) through directed differentiation. In particular, the invention disclosed herein relates to methods and systems for human self-organizing single-rosette brain organoid formation from pluripotent stem cells. The invention disclosed herein further relates to methods and systems for promoting human self-organizing single-rosette spheroids/brain organoids expressing markers of dorsal forebrain or human self-organizing single-rosette spheroids/brain organoids expressing markers of ventral forebrain.

In certain embodiments, the present invention provides methods comprising culturing pluripotent stem cells in vitro, wherein the culturing comprises inhibiting the BMP, Wnt, TGFP, and SHH signaling pathways within the pluripotent stem cells. In some embodiments, the culturing results in differentiation of the pluripotent stem cells into SOSRS comprising neuroepithelium having a dorsal cell fate from pluripotent stem cells tissue. In such embodiments, the SOSRS are capable of growing into neurons from multiple cortical layers, express markers for neuroepithelium and radial glial cells, and generate excitatory neurons. Dorsal SOSRS develop normal cortical layer marker expression such as CTIP2, SATB2, and Reelin as well as the outer radial glial marker, HOPX.

In certain embodiments, the present invention provides methods comprising culturing pluripotent stem cells in vitro, wherein the culturing comprises inhibiting the BMP, Wnt, and TGFβ signaling pathways, and activating the sonic hedgehog (SHH) signaling pathway within the pluripotent stem cells. In some embodiments, the culturing results in differentiation of the pluripotent stem cells into SOSRS comprising neuroepithelium having a ventral cell fate from pluripotent stem cells tissue. In such embodiments, the SOSRs are capable of growing into progenitors expressing markers for neuroepithelium and radial glial cells, and that generate GABAergic interneurons. Ventral SOSRS express NKX2.1, GABA, and somatostatin.

In certain embodiments, the present invention provides methods of producing SOSRS comprising neuroepithelium 1) having a dorsal cell fate from human pluripotent stem cells comprising differentiating pluripotent stem cells in a differentiation medium consisting essentially of an effective amount of a BMP signaling pathway inhibitor, a Wnt signaling pathway inhibitor, a TGFβ signaling pathway inhibitor, and a SHH signaling pathway inhibitor, or 2) having a ventral cell fate from human pluripotent stem cells comprising differentiating said pluripotent stem cells in a differentiation medium consisting essentially of an effective amount of a BMP signaling pathway inhibitor, a Wnt signaling pathway inhibitor, a TGFβ signaling pathway inhibitor, and a SHH signaling pathway activator.

In some embodiments for such methods, the pluripotent stem cells are a two-dimensional monolayer of pluripotent stem cells. In some embodiments, the pluripotent stem cells are human embryonic stem cells or human induced pluripotent stem cells.

In some embodiments for such methods, the culturing or the differentiating is under conditions sufficient for such time as to allow the inhibitors and activators to effect differentiation of the pluripotent stem cells into the SOSRS. In some embodiments for such methods, the culturing or the differentiating is for approximately four days. In some embodiments, activating and/or inhibiting one or more signaling pathways within the pluripotent stem cells occurs over a specified temporal period.

In some embodiments for such methods, inhibiting the BMP signaling pathway comprises culturing the pluripotent stem cells with a small molecule that inhibits the BMP signaling pathway. In some embodiments, the small molecule that inhibits the BMP signaling pathway is selected from the group consisting of 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyri-midine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347). In some embodiments, the small molecule that inhibits the BMP signaling pathway is DMH1.

In some embodiments for such methods, inhibiting the Wnt signaling pathway comprises culturing the pluripotent stem cells with a small molecule that inhibits the Wnt signaling pathway. In some embodiments, the small molecule that inhibits the Wnt signaling pathway is selected from the group consisting of N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phe-nylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyri-midin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein (SFRP1) 1. In some embodiments, the small molecule that inhibits the Wnt signaling pathway is XAV939.

In some embodiments for such methods, inhibiting the TGFβ signaling pathway comprises culturing the pluripotent stem cells with a small molecule that inhibits the TGFβ signaling pathway. In some embodiments, the small molecule that inhibits the TGFβ signaling pathway is selected from the group consisting of 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinox-aline (SB525334), 2-(5-Benzo[1,3]dioxol-5-yl-2-ieri-butyl-3H-imidazol-4-yl)-6-methylpyridin-e hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-be-nzamide hydrate, left-right determination factor (Lefty), 3-(6-Methyl-2-pyridinyl)-N-phenyl-4-(4-quinolinyl)-1H-pyrazole-1-carbothi-oamide (A 83-01), 4-[4-(2,3-Dihydro-1,4-benzodioxin-6-yl)-5-(2-pyridinyl)-1H-imidazol-2-yl]-benzamide (D 4476), 4-[4-[3-(2-Pyridinyl)-1H-pyrazol-4-yl]-2-pyridinyl]-N-(tetrahydro-2H-pyra-n-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazo-le-1-ethanol (R 268712) or 2-(3-(6-Methylpyridine-2-yl)-1H-pyrazol-4-yl)-1,5-naphthyridine (RepSox). In some embodiments, the small molecule that inhibits the TGFβ signaling pathway is SB431542.

In some embodiments for such methods, inhibiting the SHH signaling pathway comprises culturing the pluripotent stem cells with a small molecule that inhibits the SHH signaling pathway. In some embodiments, the small molecule that inhibits the SHH signaling pathway is cyclopamine.

In some embodiments for such methods, wherein activating the SHH signaling pathway comprises culturing the pluripotent stem cells with a small molecule that activates the SHH signaling pathway. In some embodiments, the small molecule that activates the SHH signaling pathway is SAG.

In some embodiments for such methods, the methods further comprise transferring a portion of the cultured pluripotent stem cells onto a gelatinous basement membrane matrix.

In certain embodiments, the present invention provides compositions comprising self-organizing single-rosette spheroids (SOSRS)/brain organoids comprising neuroepithelium having a dorsal cell fate.

In certain embodiments, the present invention provides self-organizing single-rosette spheroids (SOSRS)/brain organoids comprising neuroepithelium having a ventral cell fate.

In certain embodiments, the present invention provides kits comprising self-organizing single-rosette spheroids (SOSRS)/brain organoids comprising neuroepithelium having a dorsal cell fate.

In certain embodiments, the present invention provides kits comprising self-organizing single-rosette spheroids (SOSRS)/brain organoids comprising neuroepithelium having a ventral cell fate.

In certain embodiments, the present invention provides methods for drug screening, comprising a) providing a composition comprising self-organizing single-rosette brain organoids (SOSRS) comprising neural progenitors and neurons having a ventral cell fate, wherein the SOSRS are generated with the methods described herein, b) applying a medicine to the SOSRS comprising neural progenitors and neurons having a ventral cell fate; and c) determining its effect on the SOSRS comprising neural progenitors and neurons having a ventral cell fate.

In certain embodiments, the present invention provides methods for screening test agents to identify treatment agents for a neuro-condition affecting neuronal network connectivity, synaptic function and/or synaptic activity, comprising: providing a composition comprising self-organizing single-rosette brain organoids (SOSRS) comprising neural progenitors and neurons having a ventral cell fate, wherein the SOSRS are generated with the methods described herein through use of iPSCs obtained from a patient having or suspected of having a neuro-condition; treating the SOSRS with a test agent; applying a stimulus (e.g., electrical stimulus, mechanical stimulus) to the treated SOSRS and measuring the amount of neurological effect; and comparing the measured amount of neurological effect with control levels. In some embodiments, a measured amount of neurological effect similar to the control levels indicates a beneficial effect on the specific neuro-condition tested. In some embodiments, a measured amount of neurological effect significantly different from the control levels indicates that the test agent does not treat and/or have a beneficial effect on the specific neuro-condition tested. In some embodiments, the neuro-condition is one or more selected from a neurodegenerative disorder (e.g., epilepsy), autism spectrum disorder (ASD), bipolar disorder, schizophrenia or a neurological, neuropsychological, neuropsychiatric, neurodegenerative, or neuropsychopharmacological disease.

In certain embodiments, the present invention provides methods for neuroteratogenic treatment screening, comprising a) providing a composition comprising self-organizing single-rosette brain organoids (SOSRS) comprising neural progenitors and neurons having a dorsal or ventral cell fate, wherein the SOSRS are generated with the method of Claim 1, b) inducing neuroteratogenic development of the SOSRS, c) applying a treatment to the SOSRS having neuroteratogenic development; and d) determining its effect on the SOSRS having neuroteratogenic effects. In some embodiments, inducing neuroteratogenic development of the SOSRS comprises exposing the SOSRS to Y27632 and/or blebbistatin. In some embodiments, inducing neuroteratogenic development of the SOSRS comprises exposing the SOSRS to any pharmaceutical agent known to induce neuroteratogenic effects on neural tissue upon exposure to such neural tissue. In some embodiments, the treatment is a pharmacological agent (e.g., an existing pharmacological agent or a pharmacological agent under development or a yet to be developed pharmacological agent). In some embodiments, the pharmacological agent is a chemical compound, small molecule, an antibody, nucleic acid molecule (e.g., siRNA, antisense oligonucleotide, an aptamer), or a mimetic peptide.

### BRIEF DESCRIPTION OF THE DRAWINGS

FIG. 1: SOSRS demonstrate characteristics of early cortical development and consistent growth kinetics. a Schematic of SOSRS differentiation timeline. The top half describes the media components while the bottom half is the culture format. b-e Phase micrographs of SOSRS at important stages including neuroepithelial monolayer (b), monolayer cutting (c), early SOSRS formation (d), and a SOSRS grown for an additional week (e). f,g Confocal microscopy of whole mount day 8 SOSRS were immunostained for the designated proteins; nuclei were stained with bis-benzamide (DNA). h Three iPSC lines were grown in the Incucyte live-cell imaging system. The average diameter for all SOSRS for each line were plotted and used to generate the slope and R². FF-H11: n=11; FF-E9: n=10; AICS-0023: n=60. i-n Confocal micrographs of SOSRS immunostained for the designated proteins; nuclei were stained with bis-benzamide (DNA). Days of differentiation are indicated in the upper right corner of each image. All scale bars are 100 μM.

FIG. 2: Day 13 SOSRS treated with either vehicle (upper panels) or 3 μM CHIR99021 (lower panels). SOSRS were stained for cleaved-caspase and nuclei with bis-benzamide. Scale bars are 100 μM.

FIG. 3: SOSRS have neurodevelopmentally consistent layering. a,b SOSRS immunostained for the general radial glial marker PAX6 and intermediate progenitor marker TBR2. c,d Traces of evoked action potential trains c and spontaneous activity d for whole cell current-clamp recordings on neurons grown for 2 weeks from 3-month dissociated SOSRS. The inset magnifies the first action potential. e-h SOSRS at several timepoints immunostained for Reelin and either Laminin-alpha1 or HOPX. i-k, n SOSRS immunostained for cortical layer markers CTIP2, SATB2, BRN2, and CUX1. l 8 week SOSRS begin to express the outer radial glial marker HOPX with radial oriented processes. m SOSRS at 5 months express the astrocyte marker GFAP on the outer edge and mature neuronal marker MAP2ab throughout. ZO1 depicted in white was from the ZO1-EGFP fusion reporter. Scale bars are 100 μM.

FIG. 4: Single cell RNA sequencing of SOSRS. a UMAP overlay of four one-month SOSRS color coded as shown on the right. b UMAP plot with 7 identified clusters color coded. c Bar graph for 4 one-month SOSRS showing the percentage of cells found in each cluster. The legend right has the cell type identity for each cluster. d UMAP overlay of six three-month SOSRS color coded as shown on the right. e UMAP plot with 7 identified clusters color coded. f Bar graph for the 6 three-month SOSRS showing the percentage of cells found in each cluster. The legend on the right has the cell type identity for each cluster. g-1 Individual UMAP plots of each of the 6 three-month SOSRS.

FIG. 5: SAG efficiently patterns ventral SOSRS. a Schematic depicting the differentiate protocol for ventral SOSRS with variations from the dorsal cortical method in bold. b-d Sectioned day 30 ventral SOSRS immunostained for the ventral telencephalic marker, NKX2.1, pan-telencephalic marker FOXG1, and interneuron markers somatostatin (SST) and GABA. e-f Day 60 ventral SOSRS were immunostained for NKX2.1, GABA, and the mature neuronal marker, MAP2ab. g Growth curve comparison between 3 batches of dorsal SOSRS and 2 batches of ventral SOSRS. Error bars in SEM. All scale bars are 100 μM.

FIG. 6: Single cell RNA sequencing data from 1-month and 3-month SOSRS. a Replicate one-month SOSRS UMAP with color coded clusters found in FIG. 4b for comparison. b-g Additional one-month SOSRS marker heat mapped UMAP scatter plots. h-1 Cell cycle marker heat maps and corresponding violin plots demonstrating cluster 7 is made up of cells in S and M phases. m Replicate three-month SOSRS UMAP with color coded clusters found in FIG. 4e for comparison. n-s Additional three-month SOSRS marker heat mapped UMAP scatter plots. t-y Astrocyte marker heat maps for the three-month SOSRS data few astrocytes at this timepoint.

FIG. 7: A,B Confocal images of SOSRS treated and untreated with rho-kinase inhibitor, Y-27632 from 5-7 days of differentiation. C ZO-1, a tight junction marker, was used to measure the surface area of the apical end-feet. D,E Epifluorescence images of SOSRS treated and untreated with the non-muscle myosin inhibitor, blebbistatin. F Ratios of lumen/total area for each SOSRS. G Circularity of lumen for each of the SOSRS. **=p<0.01, ***=p<0.001, and ****=p<0.0001 by t-test.

FIG. 8: SOSRS generated from a ZO-1-EGFP reporter line live-imaged with an overlay of phase and EGFP channels. The EGFP shows a clear lumen structure forming as early as day 4. Scale bars are 100 μM.

FIG. 9: A,B Representative images of day 6 SOSRS treated for 24 hours with vehicle (DMSO) A or 200 μM valproic acid B. The ZO1-EGFP fusion protein is in green and bis-benzamide in blue. C,D Quantification of SOSRS lumen/SOSRS area ratio as measured using the green/blue images represented in A,B. C Shows data separated by experimental batch denoted by the number (1-3). D Combined data across the 3 experiments. Kruskal-Wallis test with Dunn's corrected post hoc test. ****=p<0.0001. Size bars are 100 μM.

FIG. 10: SOSRS automated live-imaging and analysis masking. A Brightfield, B mCherry, C Phase/brightfield SOSRS analysis mask, D Orange fluorescence SOSRS analysis mask for mCherry. Scale bars=400 μM.

FIG. 11: A, B, Day 17 SOSRS were frozen, thawed and cultured until day 30 (B) or maintained in culture without freezing (A). The morphologies were similar. C, Growth rate comparison between thawed vitrified SOSRS and controls for both dorsal (larger) and ventral (smaller) SOSRS. Error bars are SEM. Dorsal control n=23; dorsal vitrified n=3; ventral control n=16; ventral vitrified n=4. Scale bars are 100 μM.

### DEFINITIONS

As used herein, the term “pluripotent stem cells (PSCs),” also commonly known as PS cells, encompasses any cells that can differntiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryonic stem cells (including embryonic germ cells) or obtained through induction of a non-pluripotent cell, such as an adult somatic cell, by forcing the expression of certain genes.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cell and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As used herein, the term “organoid” is used to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc.

### DETAILED DESCRIPTION OF THE INVENTION

The field of brain organoid research is complicated by structural variability with multiple rosette structures per organoid. Utilizing an extracellular matrix 3D-culture-method, experiments conducted during the course of developing embodiments for the present invention resulted in the development of a new brain organoid technique that generates self-organizing, single-rosette spheroids (SOSRS) with a reproducible diameter. SOSRS maintain a single-rosette structure throughout development and can be patterned to either dorsal or ventral cell fates by adding cyclopamine or SAG, respectively. Novel aspects of this technique include beginning with a 2-dimensional monolayer of human pluripotent stem cells (hPSCs) that are patterned with small molecules into neuroepithelium rather than patterning an hPSC-derived embryoid body. This follows the normal neurodevelopmental transition from 2-dimensional neural plate to 3-dimensional neural tube. Within 8 days of differentiation, the vast majority (>99%) of monolayer fragments form spheres with a single central lumen. Dissociated neurons from 3-month-old SOSRS demonstrated spontaneous action potential firing and repetitive evoked action potentials. Additional experiments utilized this model to evaluate the teratogenicity of drugs or genetic pathways associated with neural tube defects (NTDs), and found that the SOSRS rapidly model drug-induced NTDs. The human SOSRS model represents a powerful approach for investigating mechanisms of genetic neurodevelopmental disorders and for therapeutic drug and neurotoxicity screening.

Specifically, such experiments resulted in the generation of SOSRS that generate excitatory cortical-like neurons. A key technical difference was starting with a 2-dimensional monolayer of pluripotent stem cells (PSCs) that were patterned with small molecules into neuroepithelium rather than patterning a 3-dimensional sphere of human PSCs termed an embryoid body. To pattern the 2-dimensional monolayer, such experiments utilized a cocktail of four small molecule inhibitors (DMH1, XAV939, SB431542, and cyclopamine) in N2B27 culture medium. After 4 days of patterning, this monolayer was cut into uniformly sized pieces using the StemPRO EZPASSAGE tool (Thermo Fisher). The pieces were then passaged onto solidified, undiluted Geltrex (Thermo Fisher), a gelatinous basement membrane matrix. Within 4 days, the vast majority of monolayer fragments formed spheres with a single central lumen. At this time, the cells in these spheres labeled with markers for neuroepithelium and radial glial cells, the stem cells of the developing cortex. The SOSRS were shown to have a consistent diameter and rapid growth, producing neurons from multiple cortical layers. It was also shown that replacement of cyclopamine with the sonic hedgehog pathway activator, SAG, generated SOSRS expressing markers of ventral forebrain. Over time, these SOSRS were shown to generate a large number of GABAergic (inhibitory) interneurons.

Taken together, such experiments demonstrate an efficient and robust in vitro system to generate human self-organizing single-rosette spheroids/brain organoids expressing markers of dorsal forebrain or human self-organizing single-rosette spheroids/brain organoid expressing markers of ventral forebrain

Accordingly, the invention disclosed herein generally relates to methods and systems for converting stem cells into specific tissue(s) or organ(s) through directed differentiation. In particular, the invention disclosed herein relates to methods and systems for human self-organizing single-rosette spheroid/brain organoid formation from pluripotent stem cells. The invention disclosed herein further relates to methods and systems for promoting human self-organizing single-rosette spheroids/brain organoids expressing markers of dorsal forebrain (e.g., normal cortical layer marker expression such as CTIP2, SATB2, BRN2, CUX1 and Reelin as well as the outer radial glial marker, HOPX) or human self-organizing single-rosette spheroids/brain organoids expressing markers of ventral forebrain (e.g., NKX2.1, GABA, and somatostatin).

In some embodiments, an important step is to obtain stem cells that are pluripotent or can be induced to become pluripotent.

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing neural tissues or precursors thereof, germ cells may be used in place of, or with, the stem cells to provide at least one cerebral organoid, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (available on the World Wide Web at subdomain stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl, Acad. Sci. USA 95:13726, 1998) may also be used in the methods disclosed herein. mESCs, are described, for example, by Tremml et al (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr, Top. Dev. Biol. 38:133 ff., 1998) and Reubinoff et al., Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (.sup.about.200 U/mL; Gibco) or by selection of individual colonies by micropipette. Clump sizes of about 50 to 100 cells are optimal.

Briefly, genital ridges processed to form disaggregated cells. EG growth medium is DMEM, 4500 mg/L D-glucose, 2200 mg/L mM NaHCO.sub.3; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10.mu.M forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad y-irradiation.sup.about.0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one differentiation medium and/or agent according to the methods as disclosed herein, whereas in other examples it may be desirable to differentiate the stem cells to one or more intermediate cell types prior to exposure of the at least one differentiation medium or agent described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

In another embodiment, pluripotent cells are present in embryoid bodies and are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, an important step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo.

In some embodiments, PSCs, such as ESCs and iPSCs, undergo directed differentiation in a step-wise manner into human self-organizing single-rosette spheroids (SOSRS)/brain organoids. In some embodiments, the SOSRS comprise neural progenitor cells having either a dorsal cell fate or a ventral cell fate. In some embodiments, the pluripotent stem cells are a 2-dimensional monolayer of pluripotent stem cells.

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, or described generally, in the current edition of Culture of Animal Cells: A Manual of Basic Technique (R. I. Freshney ed., Wiley & Sons); General Techniques of Cell Culture (M. A. Harrison & T. F. Rae, Cambridge Univ, Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM .beta.-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Pluripotent SCs can be maintained in an undifferentiated state even without feeder cells. The environment for feeder-free cultures includes a suitable culture substrate, particularly an extracellular matrix such as MATRIGEL® or laminin. Typically, enzymatic digestion is halted before cells become completely dispersed (about 5 min with collagenase IV). Clumps of about 10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (about 4,000 rad) primary mouse embryonic fibroblasts, telomerized mouse fibroblasts, or fibroblast-like cells derived from pPS cells. Medium can be conditioned by plating the feeders at a density of about 5-6.times.104 cm.sup.-2 in a serum free medium such as KO DMEM supplemented with 20% serum replacement and 4 ng/mL bFGF. Medium that has been conditioned for 1-2 days is supplemented with further bFGF, and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present in human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

As such, in some embodiments, methods are provided for the directed differentiation of pluriopotent cells (e.g., iPSCs or ESCs) into human SOSRS. In some embodiments, the SOSRs comprise neuroepithelium having either a dorsal cell fate or a ventral cell fate. In some embodiments, the pluripotent stem cells are a 2-dimensional monolayer of pluripotent stem cells.

Such methods are not limited to a particular manner of accomplishing the directed differentiation of PSCs into human SOSRS comprising neuroepithelium having either a dorsal cell fate or a ventral cell fate. Indeed, any method for producing such human SOSRS comprising neuroepithelium having either a dorsal cell fate or a ventral cell fate from pluripotent cells (e.g., iPSCs or ESCs) is applicable to the methods described herein. In some embodiments, the pluripotent stem cells are a 2-dimensional monolayer of pluripotent stem cells. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce human SOSRS comprising neural progenitors and neurons having either a dorsal cell fate or a ventral cell fate. In some embodiments, iPSCs are used to produce human SOSRS comprising neural progenitors and neurons having either a dorsal cell fate or a ventral cell fate.

Indeed, the SOSRS described herein can be produced according to any suitable culturing protocol to differentiate a pluripotent stem cell to a desired stage of differentiation. In some embodiments, the pluripotent cell is a human cell. In some embodiments, the pluripotent cell is not a human cell. In certain aspects, the pluripotent cell is a mouse cell. In some embodiments, the SOSRS is produced by culturing at least one stem cell for a period of time and under conditions suitable for the at least one stem cell to differentiate into the neural tissue or a precursor thereof. Such techniques are not limited to a particular manner of inducing formation of human SOSRS comprising neural progenitors and neurons having a dorsal cell fate from pluripotent stem cells. In some embodiments, inducing formation of human SOSRS comprising n neural progenitors and neurons having a dorsal cell fate from pluripotent stem cells is accomplished through selectively inhibiting the BMP, Wnt, TGFP, and sonic hedgehog (SHH) signaling pathways. In some embodiments, inhibiting the BMP, Wnt, TGFP, and SHH signaling pathways within the pluripotent stem cells comprises culturing the pluripotent stem cells with a BMP signaling pathway inhibitor, a Wnt signaling pathway inhibitor, a TGFβ signaling pathway inhibitor, and a SHH signaling pathway inhibitor. In some embodiments, inhibiting the BMP, Wnt, TGFP, and SHH signaling pathways within the pluripotent stem cells comprises culturing the pluripotent stem cells with a differentiation composition comprising a BMP signaling pathway inhibitor, a Wnt signaling pathway inhibitor, a TGFβ signaling pathway inhibitor, and a SHH signaling pathway inhibitor. In some embodiments, inhibiting the BMP, Wnt, TGFP, and SHH signaling pathways within the pluripotent stem cells comprises culturing the pluripotent stem cells with DMH1, XAV939, SB431542, and cyclopamine.

Such techniques are not limited to a particular manner of inducing formation of human SOSRS comprising neural progenitors and neurons having a ventral cell fate from pluripotent stem cells. In some embodiments, inducing formation of human SOSRS comprising neural progenitors and neurons having a ventral cell fate from pluripotent stem cells is accomplished through selectively inhibiting the BMP, Wnt, and TGFβ signaling pathways, and activating the SHH signaling pathway. In some embodiments, inhibiting the BMP, Wnt, and TGFβ signaling pathways, and activating the SHH signaling pathway within the pluripotent stem cells comprises culturing the pluripotent stem cells with a BMP signaling pathway inhibitor, a Wnt signaling pathway inhibitor, a TGFβ signaling pathway inhibitor, and a SHH signaling pathway activator. In some embodiments, inhibiting the BMP, Wnt, and TGFβ signaling pathways, and activating the SHH signaling pathway within the pluripotent stem cells comprises culturing the pluripotent stem cells with a differentiation composition comprising a BMP signaling pathway inhibitor, a Wnt signaling pathway inhibitor, a TGFβ signaling pathway inhibitor, and a SHH signaling pathway activator. In some embodiments, inhibiting the BMP, Wnt, TGFβ signaling pathways and activating the SHH signaling pathway within the pluripotent stem cells comprises culturing the pluripotent stem cells with DMH1, XAV939, SB431542, and smoothened agonist (SAG).

In some embodiments, the embryonic stem cells or iPSCs are treated with such signaling pathway activators or inhibitors for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; or 240 or more hours.

In some embodiments, the embryonic stem cells or iPSCs are treated with such signaling pathway activators or inhibitors at a concentration of 10 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher. In some embodiments, concentration of such signaling pathway activators or inhibitors within the differentiation composition is maintained at a constant level throughout the treatment. In other embodiments, concentration of such signaling pathway activators or inhibitors within the differentiation composition is varied during the course of the treatment. In some embodiments, such signaling pathway activators or inhibitors within the differentiation composition are suspended in media that include fetal bovine serine (FBS) with varying HyClone concentrations. One of skill in the art would understand that the regimen described herein is applicable to any known growth factors, alone or in combination. When two or more growth factors are used, the concentration of each growth factor may be varied independently.

In some embodiments, the obtained human SOSRS comprising neural progenitors and neurons having either a dorsal cell fate or a ventral cell fate are capable of generating excitatory cortical like neurons.

In some embodiments, selective inhibiting of the BMP signaling pathway is accomplished with a small molecule or antagonist that inhibits the BMP signaling pathway. BMPs bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (SMAD), resulting in a signal transduction pathway leading to transcriptional activity.

A BMP inhibitor (e.g., a small molecule or antagonist that inhibits the BMP signaling pathway) is defined as an agent that binds to a BMP molecule to form a complex wherein the BMP activity is neutralized, for example by preventing or inhibiting the binding of the BMP molecule to a BMP receptor. Alternatively, said inhibitor is an agent that acts as an antagonist or reverse agonist. This type of inhibitor binds with a BMP receptor and prevents binding of a BMP to said receptor. An example of a latter agent is an antibody that binds a BMP receptor and prevents binding of BMP to the antibody-bound receptor.

A BMP inhibitor may be added to iPSCs for purposes of directed differentiation of such cells toward human SOSRS comprising neural progenitors and neurons having either a dorsal cell fate or a ventral cell fate. In some embodiments, the amount of BMP inhibitor added to iPSCs for purposes of directed differentiation of such cells toward human SOSRS comprising neural progenitors and neurons having either a dorsal cell fate or a ventral cell fate is any amount effective to inhibit a BMP-dependent activity in such cells to at most 90%, more preferred at most 80%, more preferred at most 70%, more preferred at most 50%, more preferred at most 30%, more preferred at most 10%, more preferred 0%, relative to a level of a BMP activity in the absence of said inhibitor, as assessed in the same cell type. As is known to a skilled person, a BMP activity can be determined by measuring the transcriptional activity of BMP, for example as exemplified in Zilberberg et al., 2007. BMC Cell Biol. 8:41.

Several classes of natural BMP-binding proteins are known, including Noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems).

BMP pathway inhibitors may include inhibitors of BMP signaling in general or inhibitors specific for BMP1, BMP2, BMP3, BMP4, BMP5, BMP6, BMP7, BMP8a, BMP8b, BMP10 or BMP15. Exemplary BMP inhibitors include 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyri-midine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347).

In some embodiments, the BMP inhibitor is DMH1. In some embodiments, the amount of DMH1 added to the iPSCs for purposes of directed differentiation of such cells toward human SOSRS comprising neural progenitors and neurons having either a dorsal cell fate or a ventral cell fate is, for example, at a concentration of at least 10 ng/ml, more preferred at least 20 ng/ml, more preferred at least 50 ng/ml, more preferred at least 100 ng/ml. A still more preferred concentration is approximately 100 ng/ml or exactly 100 ng/ml.

The Wnt signalling pathway is defined by a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors.

Wnt is a family of highly conserved secreted signaling molecules that regulate cell-to-cell interactions and are related to the Drosophila segment polarity gene, wingless. In humans, the Wnt family of genes encodes 38 to 43 kDa cysteine rich glycoproteins. The Wnt proteins have a hydrophobic signal sequence, a conserved asparagine-linked oligosaccharide consensus sequence (see e.g., Shimizu et al Cell Growth Differ 8: 1349-1358 (1997)) and 22 conserved cysteine residues. Because of their ability to promote stabilization of cytoplasmic beta-catenin, Wnt proteins can act as transcriptional activators and inhibit apoptosis. Overexpression of particular Wnt proteins has been shown to be associated with certain cancers.

A Wnt inhibitor herein refers to Wnt inhibitors in general. Thus, a Wnt inhibitor refers to any inhibitor of a member of the Wnt family proteins including Wnt1, Wnt2, Wnt2b, Wnt3, Wnt4, Wnt5A, Wnt6, Wnt7A, Wnt7B, Wnt8A, Wnt9A, Wnt10a, Wnt11, and Wnt16. Certain embodiments of the present methods concern a Wnt inhibitor in the differentiation composition or medium. Examples of suitable Wnt inhibitors, already known in the art, include N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phe-nylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyri-midin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein 1 (SFRP1). In addition, inhibitors of Wnt can include antibodies to, dominant negative variants of, and siRNA and antisense nucleic acids that suppress expression of Wnt. Inhibition of Wnt can also be achieved using RNA-mediated interference (RNAi).

Transforming growth factor beta (TGFP) is a secreted protein that controls proliferation, cellular differentiation, and other functions in most cells. It is a type of cytokine which plays a role in immunity, cancer, bronchial asthma, lung fibrosis, heart disease, diabetes, and multiple sclerosis. TGFβ exists in at least three isoforms called TGFβ1, TGFβ2 and TGFβ3. The TGFβ family is part of a superfamily of proteins known as the transforming growth factor beta superfamily, which includes inhibins, activin, anti-mullerian hormone, bone morphogenetic protein, decapentaplegic and Vg-1.

TGFβ pathway inhibitors may include any inhibitors of TGFβ signaling in general. For example, the TGFβ pathway inhibitor is 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinox-aline (SB525334), 2-(5-Benzo[1,3]dioxol-5-yl-2-ieri-butyl-3H-imidazol-4-yl)-6-methylpyridin-e hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-be-nzamide hydrate, left-right determination factor (Lefty), 3-(6-Methyl-2-pyridinyl)-N-phenyl-4-(4-quinolinyl)-1H-pyrazole-1-carbothi-oamide (A 83-01), 4-[4-(2,3-Dihydro-1,4-benzodioxin-6-yl)-5-(2-pyridinyl)-1H-imidazol-2-yl]-benzamide (D 4476), 4-[4-[3-(2-Pyridinyl)-1H-pyrazol-4-yl]-2-pyridinyl]-N-(tetrahydro-2H-pyra-n-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazo-le-1-ethanol (R 268712) or 2-(3-(6-Methylpyridine-2-yl)-1H-pyrazol-4-yl)-1,5-naphthyridine (RepSox).

In some embodiments, a SHH signaling pathway agonist (e.g., a small molecule or agonist that activates the SHH signaling pathway) is additionally added to the iPSCs for purposes of directed differentiation of such cells toward human SOSRS comprising neural progenitors and neurons having a ventral cell fate. In some embodiments, the hedgehog signaling pathway agonist is any compound that activates the hedgehog receptor. In some embodiments, the hedgehog signaling pathway agonist is smoothened agonist (SAG).

In some embodiments, a SHH signaling pathway inhibitor (e.g., a small molecule or agonist that activates the SHH signaling pathway) is additionally added to the hESCs or iPSCs for purposes of directed differentiation of such cells toward human SOSRS comprising neural progenitors and neurons having a dorsal cell fate. In some embodiments, the hedgehog signaling pathway agonist is any compound that inhibits the hedgehog receptor. In some embodiments, the hedgehog signaling pathway inhibitor is cyclopamine.

In certain embodiments, the human SOSRS comprising neural progenitors and neurons having a dorsal cell fate or ventral cell fate produced in vitro from the described methods can be used for a number of important research, development, and commercial purposes.

In some embodiments, the methods disclosed herein result in a cell population of at least or about 10⁶, 10⁷, 10⁸, 5×10⁸, 10⁹, 10¹⁰ cells (or any range derivable therein) comprising at least or about 90% (for example, at least or about 90%, 95%, 96%, 97%, 98%, 99%, 99.5%, or any range derivable therein) human SOSRS comprising neural progenitors and neurons having a dorsal cell fate or ventral cell fate.

In some embodiments, starting cells for the present methods may comprise the use of at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

The invention also provides methods of modeling diseases involving neural tissue. In some embodiments, the modeling comprises generating SOSRS from induced pluripotent stem cells (iPSCs) derived from a patient with a disease or related disease to be modeled. In some cases, the disease is a neurological disease, but the type of disease is not limited. In some cases, the disease is a neuropsychiatric disease.

In some embodiments, the modeling comprises generating SOSRS by methods disclosed herein and inducing a disease or disease-like state. The disease or disease-like state may be induced through any method known in the art. For example, induction may be via a chemical or biological agent such as a virus, neurotoxin, bacteria, metal, small molecule, peptide or polynucleotide. The brain organoid for modeling may also be produced by any known genetic engineering technique. In some embodiments, the brain organoid may be produced from cells having one or more modified genes or genetic locus. For example, the cells may have one or more genes partially or fully deleted or partially or fully added. The brain organoids used of modeling may be cultured for any period including about 1, 2, 3, 4, 5, 6, 7, 8, 9, 10, 11, 12, 13, 14, 15, 16, 17, 18, 19, 20, 21, 22, 23 or 24 months. In some embodiments, the SOSRS are cultured for about 9 months. In some embodiments, the brain organoid is cultured for about 9-13 months.

In some embodiments, the methods of modeling may include co-culturing the SOSRS with any other cell type. The other cell types include, but are not limited to, one or more of microglia, oligodendrocytes, endothelial cells, meningeal cells, cells of the immune system and stromal cells. In some embodiments, the modeling comprises co-culture of SOSRS with microglia or other cell types transplanted into the SOSRS. In some embodiments synaptic pruning affected in psychiatric disease are modeled by co-culturing with, for example, microglia transplanted into the organoid.

The invention provides a method of screening patients with a neuro disorder or neuro disease or any disease affecting synaptic function, neuronal network activity and stimulation, through the generation of SOSRS from patient derived induced pluripotent stem cells (iPSCs). In some aspects, SOSRS are generated from iPSCs genetically engineered to carry mutations associated with one or more neuro disorders. A subject or patient can be one who has been previously diagnosed with or identified as suffering from or having a condition, disease, neuro disease or neuro disorder described herein in need of treatment or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors or symptoms for a condition or one or more complications related to a condition. A “subject in need” of treatment for a particular condition can be a subject having that condition, diagnosed as having that condition, or at increased risk of developing that condition relative to a given reference population.

In some embodiments, the methods described herein comprise selecting a subject diagnosed with, suspected of having, or at risk of developing a neuro disorder or neuro disease as described herein.

In some aspects, patient derived neural tissue is screened for a neuro disease and/or for pathology of neuronal network connectivity, synaptic function and synaptic activity. In some aspects, the disease may be depression, obsessive-compulsive disorder, schizophrenia, visual hallucination, auditory hallucination, eating disorder, bipolar disorder, epilepsy, autism spectrum disorder (ASD), amyotrophic lateral sclerosis (ALS) and any disease affecting neuronal network connectivity, synaptic function and synaptic activity.

The present invention contemplates methods in which neural tissue is generated according to the methods described herein from iPS cells derived from cells extracted or isolated from individuals suffering from a disease (e.g., a neuropsychiatric disease, such as epilepsy, autism spectrum disorder (ASD), schizophrenia, bipolar disorder), and that neural tissue is compared to normal neural tissue from healthy individuals not having the disease to identify differences between the generated neural tissue and normal neural tissue which could be useful as markers for disease (e.g., neuropsychiatric). In some embodiments, the iPS cells and/or neural tissue derived from neuropsychiatric patients are used to screen for agents (e.g., agents which are able to modulate genes contributing to a neurospychiatric phenotype).

In some embodiments, the invention provides a method of screening test agents to identify treatment agents for a neuro disease or diseases affecting neuronal network connectivity, synaptic function and/or synaptic activity. In some aspects, SOSRS exhibiting features of a neuro disease are generated as described by the methods of the invention. The SOSRS may be generated from iPSCs obtained from a patient having a neuro disease (e.g., a neurodegenerative disorder, such as epilepsy, autism spectrum disorder (ASD), bipolar disorder, schizophrenia or a neurological, neuropsychological, neuropsychiatric, neurodegenerative, or neuropsychopharmacological disease). In certain embodiments, the neural tissue is treated with a test agent. Any type or kind of stimulus (e.g., mechanical, chemical, electrical, physical) may be applied to the treated neural tissue and the results measured and recorded. The stimulus results of the treated tissue may be compared to control levels. In certain embodiments, stimulus results of the treated neural tissue are similar to the control levels, exhibiting the beneficial effects of the test agent on a neuro disorder. In alternative embodiments, stimulus results of the treated neural tissue are significantly different from the control levels, demonstrating that the test agent does not treat the specific neuro disorder tested.

The human SOSRS comprising neural progenitors and neurons having a dorsal cell fate or ventral cell fate produced by the methods disclosed herein may be used in any methods and applications currently known in the art for neural cells. For example, a method of assessing a compound may be provided, comprising assaying a pharmacological or toxicological property of the compound on the neural cell. There may also be provided a method of assessing a compound for an effect on a neural cell, comprising: a) contacting the neural cells provided herein with the compound; and b) assaying an effect of the compound on the neural cells.

Human SOSRS comprising neural progenitors and neurons having a dorsal cell fate or ventral cell fate can be used commercially to screen for factors (such as solvents, small molecule drugs, peptides, oligonucleotides) or environmental conditions (such as culture conditions or manipulation) that affect the characteristics of such cells and their various progeny. For example, test compounds may be chemical compounds, small molecules, polypeptides, growth factors, cytokines, or other biological agents.

In one embodiment, a method includes contacting a human SOSRS with a test agent and determining if the test agent modulates activity or function of neural cells within the population. In some applications, screening assays are used for the identification of agents that modulate neural progenitor cell proliferation or alter neural progenitor cell differentiation. Screening assays may be performed in vitro or in vivo. Methods of screening and identifying brain-related agents or neural agents include those suitable for high-throughput screening. For example, neural cells can be positioned or placed on a culture dish, flask, roller bottle or plate (e.g., a single multi-well dish or dish such as 8, 16, 32, 64, 96, 384 and 1536 multi-well plate or dish), optionally at defined locations, for identification of potentially therapeutic molecules. Libraries that can be screened include, for example, small molecule libraries, siRNA libraries, and adenoviral transfection vector libraries.

Other screening applications relate to the testing of pharmaceutical compounds for their effect on neural tissue maintenance or repair. Screening may be done either because the compound is designed to have a pharmacological effect on the cells, or because a compound designed to have effects elsewhere may have unintended side effects on cells of this tissue type.

Other embodiments can also provide use of human SOSRS comprising neural progenitors and neurons having a dorsal cell fate or ventral cell fate to enhance brain tissue maintenance and repair for any condition in need thereof, including brain degeneration or significant injury.

To determine suitability of cell compositions for therapeutics administration, the cells can first be tested in a suitable animal model. In one aspect, the human SOSRS are evaluated for their ability to survive and maintain their phenotype in vivo. Cell compositions are administered to immunodeficient animals (e.g., nude mice or animals rendered immunodeficient chemically or by irradiation). Tissues are harvested after a period of growth, and assessed as to whether the pluripotent stem cell-derived cells are still present.

A number of animals are available for testing of the suitability of the human SOSRS compositions.

The human SOSRS described herein, or a pharmaceutical composition including these cells, can be used for the manufacture of a medicament to treat a condition in a patient in need thereof. The human SOSRS can be previously cryopreserved. In certain aspects, the disclosed human SOSRS are derived from iPSCs, and thus can be used to provide “personalized medicine” for patients with brain diseases. In some embodiments, somatic cells obtained from patients can be genetically engineered to correct the disease causing mutation, differentiated into desired healthy brain tissue and/or cells, and engineered to form desired brain tissue and/or cells. Such tissue and/or cells can be used to replace the endogenous degenerated tissues and/or cells of the same patient. Alternatively, iPSCs generated from a healthy donor or from HLA homozygous “super-donors” can be used.

Human SOSRS can be used for toxicity and efficacy screening of agents that treat or prevent the development of a neurological condition. In one embodiment, SOSRS generated according to the methods described herein is contacted with a candidate agent. The viability or structure of the SOSRS (or various cells within the SOSRS) is compared to the viability or structure of an untreated control SOSRS to characterize the toxicity of the candidate compound. Assays for measuring cell viability are known in the art, and are described, for example, by Crouch et al. (see, J. Immunol. Meth. 160, 81-8); Kangas et al. (Med. Biol. 62, 338-43, 1984); Lundm et at (Meth. Enzymol. 133, 27-42, 1986); Petty et al. (Comparison of J. Biolum. Chemilum 10, 29-34, 0.1995); and Cree et al. (Anticancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

In another embodiment, the SOSRS comprises a genetic mutation that effects neurodevelopment, activity, or function. Protein or nucleotide expression of cells within the organoid can be compared by procedures well known in the art, such as Western blotting, flow cytometry, immunocytochemistry, in situ hybridization, fluorescence in situ hybridization (FISH), ELISA, microarray analysis, RT-PCR, Northern blotting, or colorimetric assays, such as the Bradford Assay and Lowry Assay.

In another embodiment, one or more candidate agents are added at varying concentrations to the culture medium containing SOSRS. An agent that promotes the expression of a protein/polypeptide of interest expressed in the cell is considered useful in the invention; such an agent may be used, for example, as a therapeutic to prevent, delay, ameliorate, stabilize, or treat an injury, disease or disorder characterized by a defect in neurodevelopment or neurological function. Once identified, agents of the invention may be used to treat or prevent a neurological condition. In another embodiment, the activity or function of a cell of the organoid is compared in the presence and the absence of a candidate compound. Compounds that desirably alter the activity or function of the cell are selected as useful in the methods of the invention.

In some embodiments, human SOSRS comprising neural progenitors and neurons having a dorsal cell fate or ventral cell fate produced in vitro from the described methods can be used to screen drugs for brain tissue uptake and mechanisms of transport (with endothelial cells forming a blood-brain barrier). For example, this can be done in a high throughput manner to screen for the most readily absorbed drugs, and can augment Phase 1 clinical trials that are done to study drug brain tissue uptake and brain tissue toxicity. This includes pericellular and intracellular transport mechanisms of small molecules, peptides, metabolites, salts.

In some embodiments, human SOSRS comprising neural progenitors and neurons having a dorsal cell fate or ventral cell fate produced in vitro from the described methods can be used to identify the molecular basis of normal human brain development.

In some embodiments, human SOSRS comprising neural progenitors and neurons having a dorsal cell fate or ventral cell fate produced in vitro from the described methods can be used to identify the molecular basis of congenital defects affecting human brain development.

In some embodiments, human SOSRS comprising neural progenitors and neurons having a dorsal cell fate or ventral cell fate produced in vitro from the described methods can be used to correct brain related congenital defects caused by genetic mutations. In particular, mutation affecting human brain development can be corrected using CRISPR gene editing and iPSC technology and genetically normal brain tissue produced in vitro from the described methods. In some embodiments, human SOSRS comprising neural progenitors and neurons having a dorsal cell fate or ventral cell fate produced in vitro from the described methods can be used to generate replacement tissue.

In some embodiments, human SOSRS comprising neural progenitors and neurons having a dorsal cell fate or ventral cell fate produced in vitro from the described methods can be used to generate replacement brain tissue for brain related disorders.

Various neurological conditions (e.g., neuro disorder or neuro disease) (e.g., neurodegenerative disorders or disease) (e.g., neuropsychiatric disorder or disease) may be treated or prevented by the introduction of the SOSRS obtained using the methods disclosed herein. The conditions include any condition, disease, disorder characterized by abnormal neurodevelopment and/or basic behavioral processes, including attentional and perceptual processing, executive function, inhibitory control (e.g., sensory gating), social cognition, and communication and affiliative behaviors.

As used herein, “neuro disorder” or “neuro disease” refer to neurodegenerative disorders, neuropsychiatric disorders and/or neurodevelopmental disorders. “Neuro disease” also refers to neurological, neuropsychological, neuropsychiatric, neurodegenerative, or neuropsychopharmacological diseases. Neuro disorders may be any disease affecting neuronal network connectivity, synaptic function and activity. “Neurodegenerative disorder” refers to a disease condition involving neural loss mediated or characterized at least partially by at least one of deterioration of neurons and/or neural progenitor cells. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barre syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral hemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or hemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

“Neuropsychiatric disorder” encompasses mental disorders attributable to diseases of the nervous system. Non-limiting examples of neuropsychiatric disorders include addictions, childhood developmental disorders, eating disorders, degenerative diseases, mood disorders, neurotic disorders, psychosis, sleep disorders, depression, obsessive-compulsive disorder, schizophrenia, visual hallucination, auditory hallucination, eating disorder, bipolar disorder, epilepsy, autism spectrum disorder (ASD), and amyotrophic lateral sclerosis (ALS).

In some embodiment, the human SOSRS can be used for autologous SOSRS grafts to those subjects suitable for receiving regenerative medicine. Such SOSRS or neurons obtained therein may be transplanted in combination with other neurological cells. Transplantation of the SOSRS produced by the disclosed methods can be performed by various techniques known in the art. The SOSRS or neurons obtained therein can be introduced into the target site in the form of cell suspension, adhered onto a matrix, such as extracellular matrix, or provided on substrate such as a biodegradable polymer. The SOSRS or neurons obtained therein can also be transplanted together (co-transplantation) with other cells, such as other neural cells. Thus, a composition comprising SOSRS obtained by the methods disclosed herein is provided.

In some embodiments, a diagnostic kit or package is developed to include human SOSRS comprising neural progenitors and neurons having a dorsal cell fate or ventral cell fate produced in vitro from the described methods and based on one or more of the aforementioned utilities.

Described herein are kits for practicing methods disclosed herein and for making SOSRS disclosed herein. In one aspect, a kit includes at least one SOSRS and at least one differentiation medium or agent as described herein, and optionally, the kit can further comprise instructions for converting at least one SOSRS to a population of SOSRS using a method described herein. In some embodiments, the kit comprises at least two differentiation mediums or agents. In some embodiments, the kit comprises at least three differentiation mediums or agents. In some embodiments, the kit comprises at least four differentiation mediums or agents. In some embodiments, the kit comprises at least five differentiation mediums or agents. In some embodiments, the kit comprises differentiation mediums or agents for differentiating pluripotent cells to SOSRS. In some embodiments, the kit comprises differentiation mediums or agents for differentiating pluripotent stem cells to SOSRS. In some embodiments, the kit comprises any combination of differentiation mediums or agents, e.g., for differentiating stem cells to SOSRS.

In some embodiments, the kit can be provided in a watertight or gas tight container which in some embodiments is substantially free of other components of the kit.

In some embodiments, the kit further optionally comprises information material. The informational material can be descriptive, instructional, marketing or other material that relates to the methods described herein and/or the use of a compound(s) described herein for the methods described herein.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. Throughout the examples use of the terms “we” or “our” or similar terms refers to the inventors of the technology described herein.

### Example I

This example describes the materials and methods used in the experiments described in Examples II-VII.

iPSC Culture

The iPSC lines were generated from foreskin fibroblasts as reported previously (see, Tidball, A. M. et al. Stem cell reports 9, 725-731 (2017)). The cultures were maintained on Geltrex-coated 6-well TC dishes in mTeSR1 medium. When the colonies reached ˜40% confluency, the cultures were washed once in 2 mL of PBS (without Ca²⁺/Mg²⁺) followed by incubation with 1 mL of L7 dissociation solutions (Lonza) and incubated for 2 minutes at 37 C. The solution was then replaced with mTeSR1 and scrapped with a mini cell scrapper. The solution was then pipetted up and down 3-6 times to break colonies into smaller pieces. The solution was then replated at a dilution of 1:8 onto newly Geltrex-coated dishes.

### SOSRS Differentiation

iPSC lines were passaged using Accutase (Innovative cell) and replated onto Geltrex-coated (1:50 dilution in DMEM/F12) 12-well plates at 1.5-2×105 cells/well in mTeSR1 with 10 μM rho-kinase inhibitor (Y-27632; Tocris, 1254). The medium without the inhibitor was replaced daily until the cells reach 80-100% confluency. The medium was then changed to 3N (50:50 DMEM/F12:neurobasal with N2 and B27 supplements) (see, Shi, Y., et al., Nature Neuroscience 15, 477-486 (2012)) without vitamin A with 2 μM DMH1 (Tocris, 4126), 2 μM XAV939 (Cayman Chemical, 13596), and 10 μM SB431542 (Cayman Chemical, 13031) (4 mL of medium per well). 75% of this medium was changed daily with 1 μM cyclopamine (Cayman Chemical, 11321) added beginning on day 1. On day 4, the monolayer was cut into squares using the StemPro EZ passage tool. The squares were then washed once with PBS and incubated for 2 minutes with L7 hPSC passaging solution (Lonza). After aspirating the L7 solution, the squares were sprayed off the bottom with the same culture medium 1 mL of preconditioned culture media with a P1000 micropipette. An additional 2 mL of fresh culture medium with the 4 inhibitors was added for a final volume of 3 mL. Approximately 200 μL of resuspended squares were then transfer into the wells of 96-well plates preincubated at 37° C. for 20 minutes with 30 μL of 100% Geltrex solution in each well. Up to 16 wells can be made using one monolayer well. Higher densities will result in fusion between nearby SOSRS forming multi-rosette doublets or “strings”. After 48 hours, daily 50% media changes were performed. On day 6 overall (2 days after cutting), the four inhibitors were no longer added but replaced with the 3 μM CHIR99021. After 3-4 additional days of culture, the wells were washed once with PBS and incubated for 2 minutes with L7 solution. The organoids were individually removed using the STRIPPER (Cooper Surgical) with 275 μM tips and plated individually in the wells of a low-adherence U-bottom 96-well plate with 200 μL of 3N medium without vitamin A with 3 μM CHIR99021, BDNF (20 ng/mL), and NT3 (20 ng/mL). Ideal diameters for picking the SOSRS are between 250-300 μM. Two days later half-fold media changes with 3N with vitamin A, BDNF (20 ng/mL), and NT3 (20 ng/mL) and repeated every other day. After 35 days of differentiation, 3N media with vitamin A without neurotrophic factors (BDNF and NT3) is used for half-fold media changes every other day. Around this time, SOSRS are transferred from the low-adherence 96-well plate to a low-adherence 24 well plate for a necessary increase in media volume due to SOSRS size.

### Ventral SOSRS Differentiation

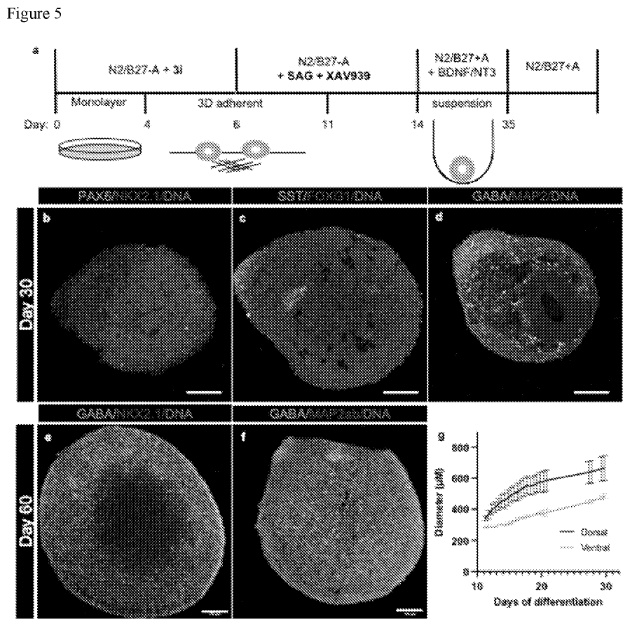

For ventral SOSRS differentiation, no cyclopamine or CHIR99021 were used. After plating onto Geltrex, 1 μM SAG and 2 μM XAV939 were added the culture media for 1 week. All other procedures were performed identically (FIG. 5).

### Immunocytochemistry

SOSRS imaged before day 11 were grown on Nunc™ Lab-Tek™ III 8-well chamber slides (Thermo Scientific) and were fixed in paraformaldehyde for 30 min at room temperature (RT). After permeabilization with 0.2% Triton-X 100 for 20 min at RT, cells were incubated in PBS containing 5% normal goat serum with 1% BSA and 0.05% Triton-X100 for 1 h at RT. Older SOSRS were fixed for 30 minutes in suspension and incubated in 30% sucrose overnight at 4 C followed by embedding in TFM medium and freezing on dry ice. The blocks were sectioned on a Cryostat with 20 μM section thickness. All samples were incubated in primary antibody overnight (see Supplemental Table 2 for antibodies and dilutions) in the same blocking buffer at 4° C., washed 4 times in PBS with 0.05% Tween-20 (PBST), and incubated for 90 min with secondary antibody. Cells were washed 3 times in PBST and incubated with bisbenzimide for 5 min. After additional PBS washes, coverslips were mounted on slides with Glycergel mounting medium (Agilent Dako). Images were obtained on a Leica SP5 upright DMI 6000 confocal microscope.

### Example II

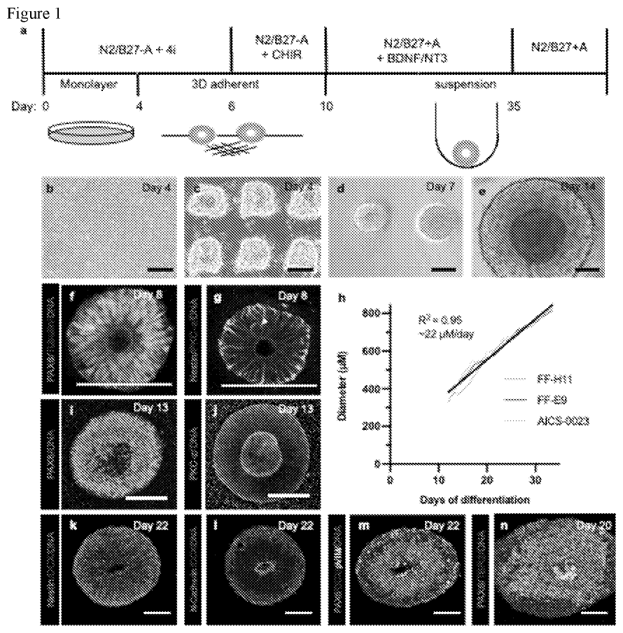

Previous 2-dimensional neuronal differentiations from iPSCs used 4 inhibitors to achieve cortical cultures¹. These inhibitors include traditional dual-SMAD inhibitors including the TGFβ inhibitor SB413542 and the ALK2 specific inhibitor DMH1^{2, 3}. The WNT pathway antagonist XAV939 increases forebrain identity⁴, and cyclopamine will inhibit any endogenous SHH signaling producing more pure dorsal forebrain^{5, 6}. While most monolayer dual-SMAD inhibition techniques last 8-12 days, we (the inventors) found consistent “rolling-up” and detachment of the monolayer between days 5-7 of differentiation. Therefore, we chose day 4 to initially test converting the 2D cultures to 3D by breaking up the monolayer and replating on thick ECM (Geltrex) (FIGS. 1a and 1b). This technique has been used to induce many 3D cultures dating back to primary mammary cultures in the 1980's⁷. Within 2-4 days, a clear single lumen was observable in nearly every 3-dimensional structure (FIG. 1d). We have further refined the technique by cutting monolayer of neuroepithelium (NE) using the StemPro EZ-passage tool to reproducibly generate ˜125 μM squares of NE on day 4 (FIG. 1c). After 24 hours, the NE squares on ECM round up into ˜100-130 μM spheres that continue to expand, forming a central lumen observable by phase microscopy (FIG. 1e). Utilizing a ZO1-EGFP fusion line to label the apical tight junctions, we live imaged SOSRS formation over 6 hours showing migration of the fluoresence from the edges to a central lumen indicating a neurulation like event, which is thought to be how 2-dimensional neural rosettes form, rather than cavitation⁸.

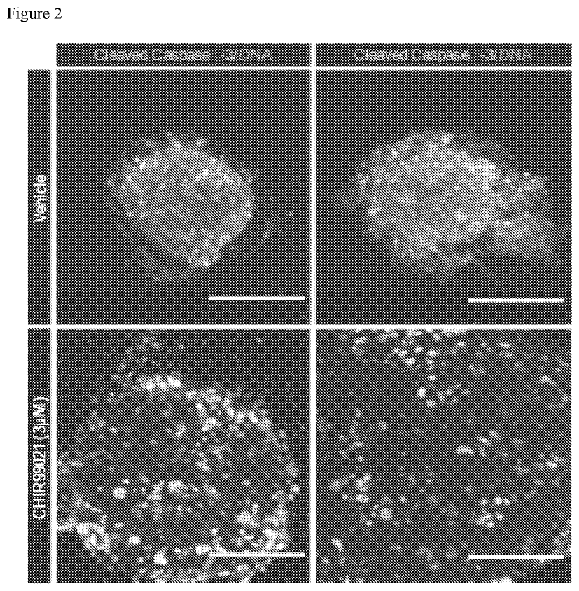

Confocal images of immunostained whole-mount day 8 SOSRS showed homogeneous neuroepithelial marker expression of PAX6 and nestin with radial organized tubulin structures (FIG. 1f). The day 8 SOSRS inner lumens label with the apical marker PKC-z (FIG. 1g), clearly demonstrating correct rosette polarization. Even at this early time, we have evidence for inter-kinetic nuclear migration based on the position of mitotic cells identified by bisbenzimide stained aligned chromosomes (FIG. 1g, arrowhead). On day 7, the SOSRS began to have significant cell death around their perimeter; likely due to the differentiation of radial glial cells to intermediate progenitors in the absence of necessary growth factors like FGF2 and EGF. The addition of the GSK3-beta inhibitor, CHIR99021, is known to stabilize beta-catenin signaling. Continued beta-catenin signaling delays the differentiation of radial glial cells into intermediate progenitor cells (IPCs)⁹. Addition of CHIR99021 from day 6-10, blocked the majority of cell death and allowed the SOSRS to grow more rapidly (FIG. 2). A similar pulse of CHIR99021 has been used previously on human brain organoids to expand rosette size and was found to increase FOXG1 expression, a definitive forebrain marker¹⁰. This pulse resulted in rapid expansion of the PAX6 positive cells while maintaining a large central lumen (FIGS. 1i and j).

To avoid projections and migration of early-born neurons extending into the Geltrex matrix, we removed the SOSRS from Geltrex on approximately day 10. We have found a diameter between 250-300 μM is optimal for cell survival and maintenance of the single rosette after removal from Geltrex. The SOSRS were transferred to low-attachment 96-well plates for suspension culture with CHIR99021 included for the first 24 hours to increase survival. Neurotrophins BDNF and NT3 were also added to the culture medium from this time until approximately 35 days of differentiation because TrkB and TrkC are the major neurotrophin receptors in the early neural tube¹¹. This overlap of CHIR99021 and neurotrophic factors was necessary for reducing cell death immediately following removal for Geltrex. These 96-well plates allowed for the isolation of individual rosettes to avoid unintentional fusion and were imaged daily using the Incucyte automated imaging platform. This allowed for easy monitoring of organoid size longitudinally, demonstrating that the growth kinetics of SOSRS is remarkably reproducible between lines and batches (FIG. 1h).

### SOSRS Demonstrate Characteristic Layering of Cortical Development

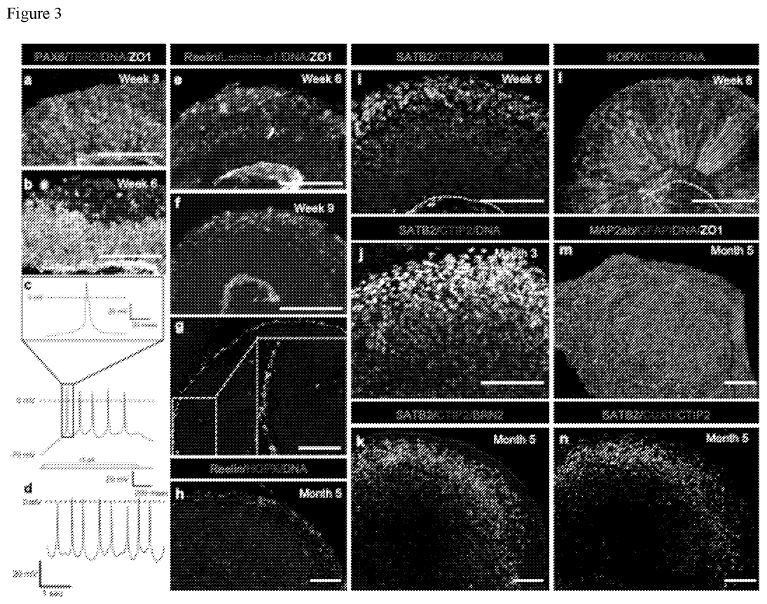

Day 22 SOSRS have clear neurogenesis as shown by the expression of Turbo-RFP expressed under the DCX promoter, with both expressed in an evenly distributed outer layer on the SOSRS surface (FIG. 1k,l,m). An apparent ventricular zone (VZ) persists around the lumen that is PAX6 positive with phospho-vimentin positive dividing radial glial cells on the lumen surface (FIG. 1m). A second proliferative zone exists on the outside of the organoid and are likely dividing IPCs (FIG. 1m). The IPC marker TBR2 labels a ring of cells in this same relative position (FIG. 1n). Older SOSRS continue to show a clear layer of TBR2 expressing cells just outside the PAX6 expressing VZ, similar to the TBR2+ subventricular zone (SVZ) of the developing cortex (FIG. 3a-b).

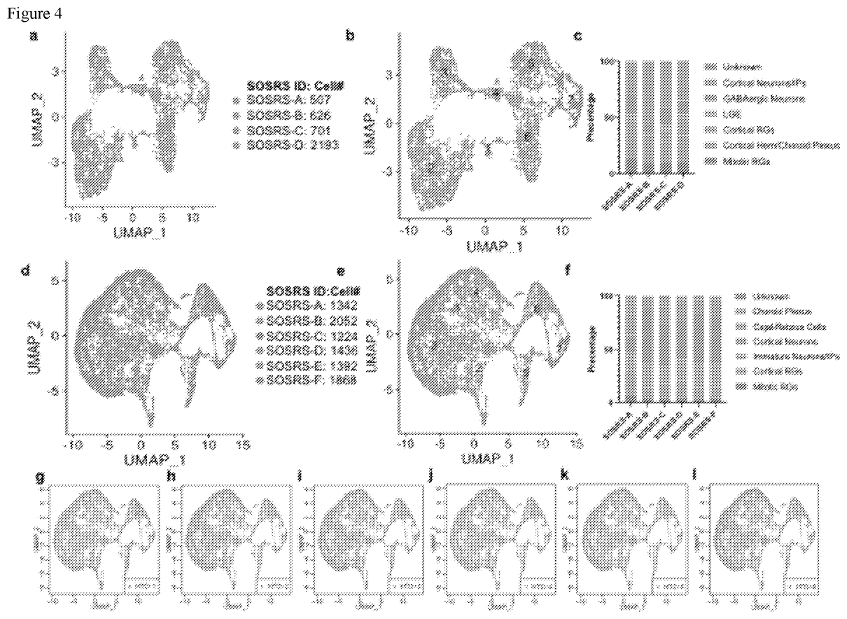

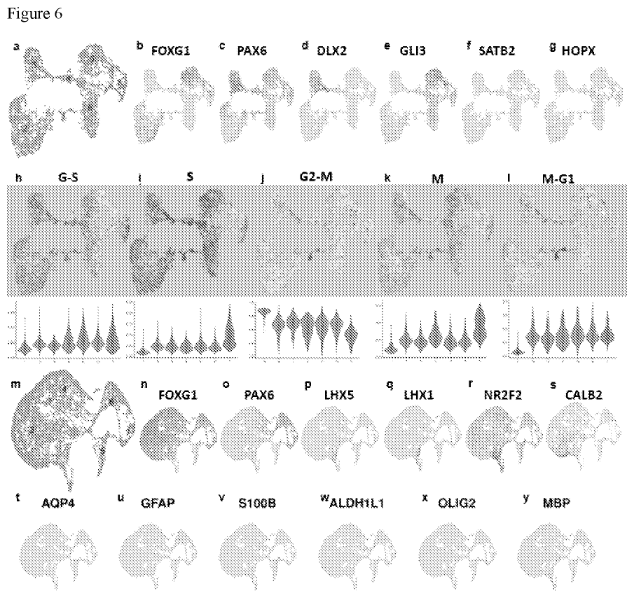

We also observe developmental layering typical of cortical development. These include the Reelin+ cells in the most superficial layer similar to the position of Reelin+ Cajal-Retzius cells in the marginal zone of the developing cortex (FIG. 3e-h). These cells continue to be present on the outer most region of SOSRS until after 5 months. We further see evidence for the cortical hem origin of Cajal-Retzius cells in the single cell RNA sequencing (scRNAseq) data for both one-month and three-month old SOSRS (FIG. 4 and FIG. 6). In addition to this marginal zone and/or layer 1 cortical marker, at 6 weeks we begin to see many cells expressing the deep layer cortical neuron marker CTIP2 superficial to the PAX6+ proliferative zone (FIG. 3i). In three-month old SOSRS, SATB2+ cells become more common superficial to the CTIP2 layer (FIG. 3j). Finally, only in 5-6 month old SOSRS do we see robust expression of the superficial cortical layer neuronal markers BRN2+ and CUX1+ on the SOSRS outer edge, peripheral to the SATB2+ cell layer (FIG. 3k,n). Therefore, our model shows typical developmentally regulated timing and positioning of cortical layer marker development. It is important to note that we only see such neurodevelopmentally normative layering in SOSRS with continued expression of Reelin+ cells on their exterior, demonstrating the importance of these cells in normal “inside-out” development of cortex. The presence of superficial neurons portends the appearance of outer radial glial cells which given rise to these neuronal subtypes in primate development. Indeed, in two-month SOSRS we see the appearance of HOPX+ cells, a quintessential marker of outer radial glia (FIG. 3l). Only sparse HOPX expression was apparent in one-month old SOSRS by scRNAseq (FIG. 6g), but was present in ˜30% of the cluster 6 radial glia in 3-month SOSRS. We observe after this time point that the SOSRS begin to become multi-rosette in nature or loss their rosette structure altogether; however, our cortical layer markers show the maintenance of a single rosette/lumen structure during the first 1-2 months of SOSRS differentiation (FIG. 1), and demonstrate that despite the loss of structure, the persistence of developmental cues, such as Reelin, still result in normal cortical layering at later time points (FIG. 3).

### Single-Cell RNA-Sequencing Shows Most Cortical Cell Types Present in SOSRS

To further characterize the cell types in our (the inventors) SOSRS and the variability between individual SOSRS, we performed scRNAseq of SOSRS differentiated for one or three months. Using the 10×genomics platform for scRNAseq and UMAP plotting, cells from 4 separate one-month SOSRS each formed 7 distinct clusters (FIG. 4b) made up of mainly radial glia (RG) (42%, clusters 5-7) and neurons (52%, cluster 2,3). In three-month-old SOSRS, there is a much greater fraction of neurons (clusters 2-4) with fewer proliferating RG (cluster 6,7). At this time, 33% of the RG express the outer radial glia marker, HOPX. The most enriched transcripts in Cluster 7 at both time points were markers of dividing cells such as MKI67. While see 25% of neurons at one-month are GABAergic (FIG. 4b cluster 3), in three-month-old SOSRS we see a near complete absence of GABAergic neurons by both scRNAseq and immunostaining. At both time points, the replicate SOSRS are remarkably similar with only one somewhat divergent SOSRS in each set (SOSRS B in FIG. 4c and SOSRS A in FIG. 4f).

Efficient Ventralization of SOSRS with SAG

The SOSRS protocol was adapted for the production of ventral telencephalon (ganglionic eminence) with cortical interneurons of the medial ganglionic eminence desired since our dorsal SOSRS lack these cells. This was accomplished with removing cyclopamine from our original protocol and adding SAG instead of CHIR99021 from day 7 to day 14 (FIG. 5a). The WNT pathway antagonist XAV939 was also continued during this time and found to increase SOSRS size and MGE specification (data not shown). Immunostaining of one-month SOSRS revealed nearly 100% NKX2.1 (a ventral telencephalic marker)/FOXG1 positive cells with a small number of early GABAergic neurons (FIG. 5b-d). Two-month ventral SOSRS contain a NKX2.1 core with GABAergic neurons surrounding (FIG. 5e,f). At this time point the rosette structure with a central lumen becomes small and decentralized due to reduced proliferative capacity of ventral SOSRS, as supported by reduced growth rate of the ventral SOSRS compared to dorsal (FIG. 5g), despite both originating from an identically sized monolayer fragment.

### Example VI. Neuroteratogen Screening

Neural tube defects (NTDs) are common congenital malformations (˜1/1000 live births in the US)¹² that can often lead to miscarriage, death, or paraplegia. These malformations can be caused by either gene variants or environmental exposures, including toxins, pharmaceuticals, and nutrient deprivation. The first pharmaceutical known to produce congenital malformations in humans was the chemotherapy drug, aminopterin¹³ that blocks the folic acid pathway. Folic acid deficiencies are the most prominent cause of congenital malformations¹⁴. The need for robust teratogen screening of novel therapeutics was highlighted by the thalidomide tragedy of the 1950's. This compound was marketed as a treatment for morning sickness during pregnancy. Although rodent testing did not indicate teratogenic risk of thalidomide use, this drug unexpectedly led to a dramatic number of congenital malformations in human fetuses including NTDs. Despite this tragedy, rodent models remain the standard method for teratogenic screening, even though the risk of species-specific differences persists. Overall, rodent models of pharmaceutical toxicity have shown a low rate of concordance with humans leading to the termination of many clinical trials due to human-specific toxicities¹⁵, and neurological toxicities are the most common cause of clinical trial termination (22%). In addition to the species-specific problems with current teratogenicity screening, these models are costly, labor intensive, and result in a moderate rate of false positives and false negatives¹⁶.

In the 1970's, a correlation between epileptic mothers and congenital malformations was identified¹⁷, but not until 2001 was it concluded that this increased risk was primarily due to anti-seizure medications (ASMs)¹⁸. Subsequently, many epidemiological studies attempted to characterize the individual teratogenic effects of each ASM¹⁹; however, such studies are complicated by differences in dosage and the high prevalence of polytherapy in epilepsy treatment. Interestingly, despite various unrelated modes of action for ASMs, most ASMs associated with teratogenicity and NTDs are thought to affect the folic acid pathway because of reduced serum folic acid and elevated homocysteine; however, supplementation with folic acid in patients taking ASMs had no significant effect on the rate of teratogenicity²⁰. Therefore, the exact mechanism by which ASMs cause NTDs is still controversial. Most expectant epilepsy mothers are advised not to discontinue ASM treatment during pregnancy due to the risk of untreated seizures to the mother and fetus, but comparative risk assessment between ASMs is minimal. Furthermore, while longstanding ASMs have been classified for neuroteratogenic risk based on epidemiology, characterizing the risk of new ASMs with an in vitro model of human neurulation could avoid needless birth defects that would only be identified in patient registries.

Human cell culture based models have shown to have more predictive value in other areas of toxicology. For example, proarrhythmic risk is now routinely assessed by iPSC-derived cardiomyocytes²¹. Human-specific models may also limit false-positives due to rodent-specific toxicities, allowing for a larger number of medications to enter clinical trials. With the advent of 3D brain organoid technology, several groups have made attempts to model neuroteratogenicity²². Unfortunately, current methods result in highly variable organoids with multiple-rosettes²². Since neural rosettes are the in vitro correlate of the developing neural tube, a multirosette model does not recapitulate normal human brain development adequately for detailed structural analyses. Therefore, nearly all models have used a transcriptomic approach to assess the neuroteratogenicity of compounds. One study used neural constructs in which they mixed nearly all cell types in the human brain derived from human stem cells to overcome the variability seen in self-organizing human brain organoids and test developmental neurotoxins²³. While they found high predictive validity of their model, it was limited by lack of a structural neurulation readout and its extremely labor-intensive nature. Our simple model of human neurulation, which we call SOSRS (self-organizing single-rosette spheroids), overcomes the lack of structural readout in previous human neural models and will likely overcome species-specific confounds. For the first time, we are able to identify NTDs generated in a human culture system.

Screening for Teratogens with SOSRS

SOSRS have clearly defined and reproducible structural morphology particularly at the earliest stages (FIGS. 1 and 3). This breakthrough has allowed us to address areas of neuroscience research not previously possible with brain organoids, including (1) easily measuring structural outcomes of treatments and (2) modeling the earliest stage of neurodevelopment, neurulation, in a uniform manner. Therefore, this system can be applied to neuroteratogenic risk where pharmaceutical or environmental compounds lead to neural tube defects. This human-specific platform could reduce both false positives that lead to unnecessary termination of lead compounds and false negatives that lead to unforeseen congenital malformations. This platform could be applied broadly to investigate potential environmental toxins, nutrient deprivations, genetic risk variants, and novel therapeutics. Similar assays have been previously developed in 2-dimensional human pluripotent stem cell systems. However, these were either entirely transcriptome based like the STOP-Tox_{UKN} system²⁴ or involved complicated machine learning algorithms to identify and measure 2-dimensional structures in the neural rosette formation assay (RoFa)²⁵. We have clearly defined structural outcomes of neuroteratogenic exposure with more simple analysis due to single rosette formation and reduced timeline with 7 total days of differentiation rather than the 15 days needed in previous studies^{24, 25}.

### Supporting Data:

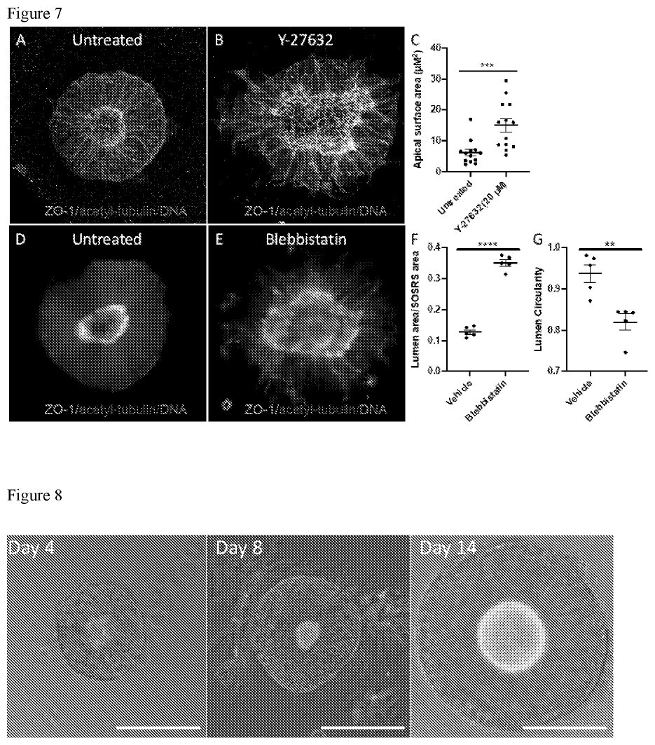

This 3D model of human neurulation allowed us to test known neuroteratogens that block a specific pathway necessary for proper neural tube formation. These were the rho-kinase inhibitor, Y-27632, and the non-muscle myosin inhibitor, blebbistatin^{26, 27}. These are key components of apical constriction of the actinomyosin cytoskeletal network. On the fifth day of differentiation and one day after plating on basement membrane matrix, the SOSRS were treated. Day 7 organoids were immunostained for the tight-junction/apical marker, ZO-1, and the stable microtubule network by acetylated-tubulin. Both compounds caused increased apical end-feet surface areas, increased lumen/total area, and decreased lumen circularity (FIG. 7).

To further increase throughput of this model of NTD formation, we have obtained an iPSC line containing an EGFP tagged TJP1, the gene that encodes ZO-1 (AICS-0023 from Allen Institute Cell line collection, obtained from Coriell Institute Biobank). This results in a green fluorescent fusion protein targeted to the apical tight junction, allowing for fluorescent imaging of the lumen structure in live cells (FIG. 8). Because our automated live-imaging platform can not image in the green channel, we have generated CRISPR gRNA and targeting plasmids to insert a TagRFP gene into the same location in one iPSC and one ESC line allowing for live timelapse imagining and automated analysis of SOSRS morphology. Therefore, once we have generated these tagged lines, we can measure NTD phenotypes in a fully human model of early neurodevelopment in an automated manner with medium throughput.

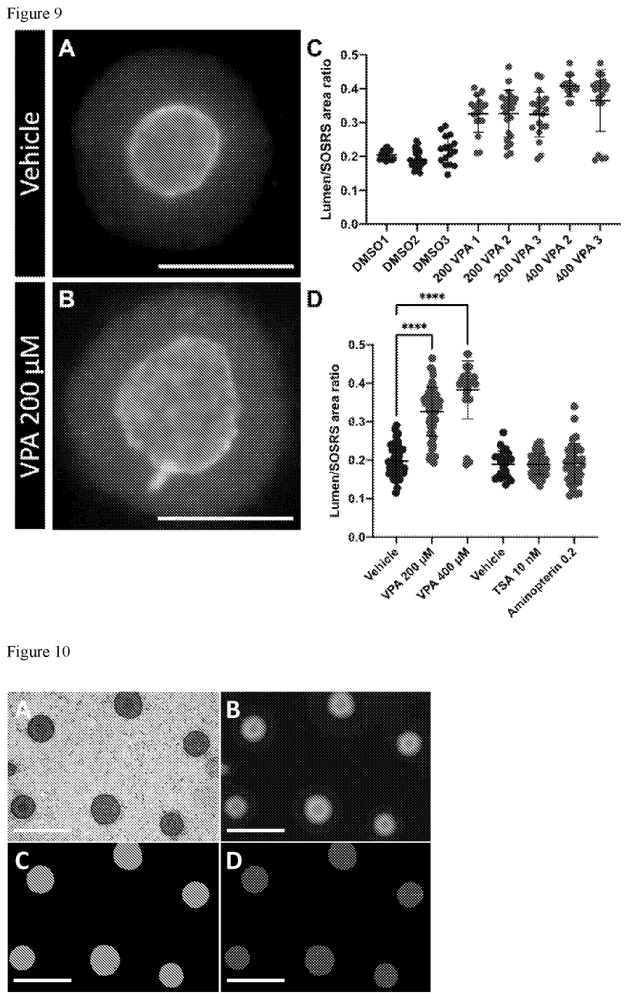

We have further tested the potential use of SOSRS for screening for neuroteratogenic effects by treating with valproic acid (VPA). VPA, an antiseizure medication, is a known neuroteratogen with an unclear mechanism. We tested 200 μM and 400 μM VPA and in each found a dramatic increase in the ratio of the lumen area to the SOSRS area (FIG. 9). This effect was not replicated with either a specific HDAC inhibitor (TSA) or folic acid pathway inhibitor (aminopterin). While our measurements and analysis for this data has been mostly manual, utilizing fluorescent proteins allows the use of live cell imaging platforms to greatly increase throughput by automating both imaging and analysis. In lieu of having the correct fluorescent protein/filter set for a ZO1 tagged line, we imaged SOSRS constitutively expressing mCherry on the IncuCyte S3 Live Cell Imaging Platform grown in 96-well format. This platform can be simultaneously scheduled for repeated imaging of up to 6 plates with automated analysis (FIG. 10). The analysis definition can work in both the phase and orange channels, with area exclusion definitions for SOSRS that are too small or too large (due to initial fragmentation or multiplets of the initial monolayer pieces). In lieu of having the ZO1-TagRFP lines to test, we utilized an iPSC line constitutively expressing cytoplasmic mCherry. The imaging and analysis for mCherry is shown in FIG. 10B,D, and similar analysis definitions will be established for lumens in ZO1-TagRFP lines. Therefore, for the first time, human neurulation can be imaged over time allowing for longitudinal studies.

### Example VII

### Cryopreservation of SOSRS Using Vitrification Technique

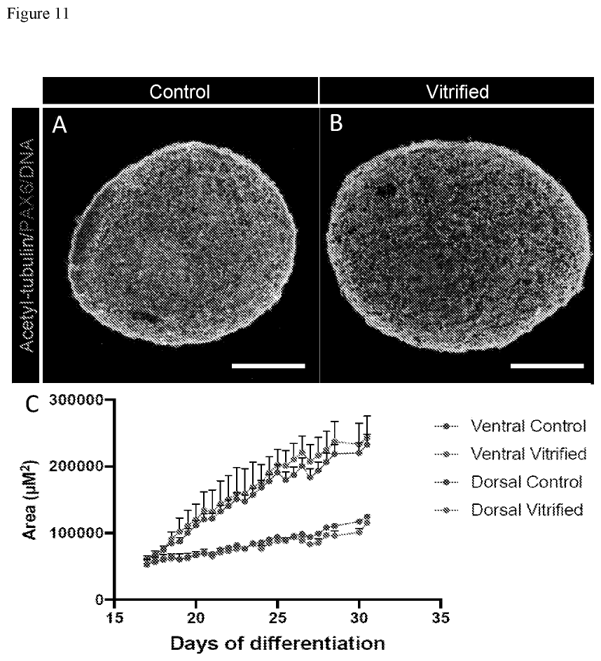

Cryopreservation at ultra-low temperature has been a valuable tool to preserve cells and tissues in different aspects of science. However, brain organoid cryopreservation remains a major challenge due to tissue complexity and the sensitivity of neurons to temperature shifts. Conventional cryopreservation techniques for stem cells and other organoid types (such as lung organoids) have not shown good results in cryopreserving brain organoids. We optimized the vitrification technique that has been extensively used previously to cryopreserve human embryos and oocytes and applied it to cryopreserve our SOSRS human brain organoid model. This technique starts with pre-equilibrating SOSRS in cryoprotectant solution, resulting in dehydration of the cells within the organoid and permeation with the cryoprotectant. The SOSRS are then exposed to a high concentration of cryoprotectant for a short period of time (˜1 min) and immediately immersed in liquid nitrogen. The high osmolality of the cryoprotectant results in complete dehydration of the cells. Using this technique, we successfully cryopreserved SOSRS at different developmental stages. We thawed organoids and successfully maintained them in culture. We examined the cytoarchitectural integrity of thawed SOSRS by immunostaining with antibodies against markers of ventricular zone radial glia and neurons. We found no significant differences between the vitrified and control SOSRS grown in parallel that were not vitrified (FIG. 11A,B). In addition, the vitrified SOSRS demonstrated a growth rate comparable to the controls (FIG. 11C). Our human SOSRS cryopreservation method provides a promising approach for bio-banking and should allow for the transfer of cryopreserved SOSRS between institutions for investigations of human brain development and genetic neurodevelopmental disorders.

### INCORPORATION BY REFERENCE

The entire disclosure of each of the patent documents and scientific articles referred to herein is incorporated by reference for all purposes. The following references, identified numerically throughout the application, are incorporated by reference:
1. Tidball, A. M. et al. Variant-specific changes in persistent or resurgent Na+ current in SCN8A-EIEE13 iPSC-derived neurons. bioRxiv, 2020.2001.2016.909192 (2020).
2. Mohedas, A. H. et al. Development of an ALK2-biased BMP type I receptor kinase inhibitor. ACS chemical biology 8, 1291-1302 (2013).
3. Chambers, S. M. et al. Highly efficient neural conversion of human ES and iPS cells by dual inhibition of SMAD signaling. Nature biotechnology 27, 275 (2009).
4. Maroof, A. M. et al. Directed differentiation and functional maturation of cortical interneurons from human embryonic stem cells. Cell stem cell 12, 559-572 (2013).
5. Gaspard, N. et al. An intrinsic mechanism of corticogenesis from embryonic stem cells. Nature 455, 351 (2008).
6. Espuny-Camacho, I. et al. Pyramidal neurons derived from human pluripotent stem cells integrate efficiently into mouse brain circuits in vivo. Neuron 77, 440-456 (2013).
7. Barcellos-Hoff, M., Aggeler, J., Ram, T. & Bissell, M. Functional differentiation and alveolar morphogenesis of primary mammary cultures on reconstituted basement membrane. Development 105, 223-235 (1989).
8. Hřibková, H., Grabiec, M., Klemová, D., Slaninová, I. & Sun, Y.-M. Calcium signaling mediates five types of cell morphological changes to form neural rosettes. Journal of cell science 131 (2018).
9. Wrobel, C. N., Mutch, C. A., Swaminathan, S., Taketo, M. M. & Chenn, A. Persistent expression of stabilized β-catenin delays maturation of radial glial cells into intermediate progenitors. Developmental biology 309, 285-297 (2007).
10. Lancaster, M. A. et al. Guided self-organization and cortical plate formation in human brain organoids. Nature biotechnology 35, 659-666 (2017).
11. Bemd, P. The role of neurotrophins during early development. Gene Expression The Journal of Liver Research 14, 241-250 (2008).
12. Mai, C. T. et al. Population-based birth defects data in the United States, 2008 to 2012: Presentation of state-specific data and descriptive brief on variability of prevalence. Birth Defects Research Part A: Clinical and Molecular Teratology 103, 972-993 (2015).
13. Warkany, J., BEAUDRY, P. H. & Hornstein, S. Attempted Abortion with Aminopterin (4-Amino-Pteroylglutamic Acid): Malformations of the Child. AMA journal of diseases of children 97, 274-281 (1959).
14. Nelson, M. M., Asling, C. W. & Evans, H. M. Production of Multiple Congenital Abnormalities in Young by Maternal Pteroylglutamic Acid Deficiency during Gestation: Thirteen Figures. The Journal of nutrition 48, 61-79 (1952).
15. Olson, H. et al. Concordance of the toxicity of pharmaceuticals in humans and in animals. Regulatory Toxicology and Pharmacology 32, 56-67 (2000).
16. Augustine-Rauch, K., Zhang, C. X. & Panzica-Kelly, J. M. A developmental toxicology assay platform for screening teratogenic liability of pharmaceutical compounds. Birth Defects Research Part B: Developmental and Reproductive Toxicology 107, 4-20 (2016).
17. Speidel, B. & Meadow, S. Maternal epilepsy and abnormalities of the fetus and newborn. The Lancet 300, 839-843 (1972).
18. Holmes, L. B. et al. The teratogenicity of anticonvulsant drugs. New England Journal of Medicine 344, 1132-1138 (2001).
19. Tomson, T. & Battino, D. Teratogenic effects of antiepileptic drugs. The Lancet Neurology 11, 803-813 (2012).
20. Hill, D. S., Wlodarczyk, B. J., Palacios, A. M. & Finnell, R. H. Teratogenic effects of antiepileptic drugs. Expert review of neurotherapeutics 10, 943-959 (2010).
21. del Álamo, J. C. et al. High throughput physiological screening of iPSC-derived cardiomyocytes for drug development. Biochimica et Biophysica Acta (BBA)-Molecular Cell Research 1863, 1717-1727 (2016).
22. Worley, K. E., Rico-Varela, J., Ho, D. & Wan, L. Q. Teratogen screening with human pluripotent stem cells. Integrative biology 10, 491-501 (2018).
23. Schwartz, M. P. et al. Human pluripotent stem cell-derived neural constructs for predicting neural toxicity. Proceedings of the National Academy of Sciences 112, 12516-12521 (2015).
24. Shinde, V. et al. Definition of transcriptome-based indices for quantitative characterization of chemically disturbed stem cell development: introduction of the STOP-Tox ukn and STOP-Tox ukk tests. Archives of toxicology 91, 839-864 (2017).
25. Dreser, N. et al. Development of a neural rosette formation assay (RoFA) to identify neurodevelopmental toxicants and to characterize their transcriptome disturbances. Archives of Toxicology 94, 151-171 (2020).
26. Escuin, S. et al. Rho-kinase-dependent actin turnover and actomyosin disassembly are necessary for mouse spinal neural tube closure. J Cell Sci 128, 2468-2481 (2015).
27. Wei, L. et al. Rho kinases play an obligatory role in vertebrate embryonic organogenesis. Development 128, 2953-2962 (2001).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method, comprising culturing pluripotent stem cells in vitro, wherein the culturing comprises inhibiting the BMP, Wnt, TGFβ, and SHH signaling pathways within the pluripotent stem cells,
wherein the culturing results in differentiation of the pluripotent stem cells into self-organizing single-rosette spheroids (SOSRS)/brain organoids comprising neural progenitors and neurons having a dorsal cell fate from pluripotent stem cells tissue.

2. The method of claim 1, wherein the pluripotent stem cells are a two-dimensional monolayer of pluripotent stem cells.

3. The method of claim 1,
wherein inhibiting the BMP signaling pathway comprises culturing the pluripotent stem cells with a small molecule that inhibits the BMP signaling pathway,
wherein inhibiting the Wnt signaling pathway comprises culturing the pluripotent stem cells with a small molecule that inhibits the Wnt signaling pathway,
wherein inhibiting the TGFβ signaling pathway comprises culturing the pluripotent stem cells with a small molecule that inhibits the TGFβ signaling pathway,
wherein inhibiting the SHH signaling pathway comprises culturing the pluripotent stem cells with a small molecule that inhibits the SHH signaling pathway.

4. The method of claim 3,
wherein the small molecule that inhibits the BMP signaling pathway is selected from the group consisting of 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyri-midine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347),
wherein the small molecule that inhibits the Wnt signaling pathway is selected from the group consisting of N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phe-nylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyri-midin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein 1 (SFRP1),
wherein the small molecule that inhibits the TGFβ signaling pathway is selected from the group consisting of 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinox-aline (SB525334), 2-(5-Benzo[1,3]dioxol-5-yl-2-ieri-butyl-3H-imidazol-4-yl)-6-methylpyridin-e hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-be-nzamide hydrate, left-right determination factor (Lefty), 3-(6-Methyl-2-pyridinyl)-N-phenyl-4-(4-quinolinyl)-1H-pyrazole-1-carbothi-oamide (A 83-01), 4-[4-(2,3-Dihydro-1,4-benzodioxin-6-yl)-5-(2-pyridinyl)-1H-imidazol-2-yl]-benzamide (D 4476), 4-[4-[3-(2-Pyridinyl)-1H-pyrazol-4-yl]-2-pyridinyl]-N-(tetrahydro-2H-pyra-n-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazo-le-1-ethanol (R 268712) or 2-(3-(6-Methylpyridine-2-yl)-1H-pyrazol-4-yl)-1,5-naphthyridine (RepSox),
wherein the small molecule that inhibits the SHH signaling pathway is cyclopamine.

5-10. (canceled)

11. The method of claim 1, wherein the culturing is for approximately four days.

12. The method of claim 1, further comprising transferring a portion of the cultured pluripotent stem cells onto a gelatinous basement membrane matrix.

13. (canceled)

14. The method of claim 1, wherein the SOSRS are capable of growing into neurons from multiple cortical layers.

15. The method of claim 1, wherein the SOSRS express markers for dorsal forebrain (e.g., normal cortical layer marker expression such as CTIP2, SATB2, and Reelin as well as the outer radial glial marker, HOPX).

16. The method of claim 1, wherein the SOSRS generate GABAergic interneurons.

17. A method, comprising culturing pluripotent stem cells in vitro, wherein the culturing comprises inhibiting the BMP, Wnt, TGFβ signaling pathways and activating the SHH signaling pathway within the pluripotent stem cells,
wherein the culturing results in differentiation of the pluripotent stem cells into self-organizing single-rosette spheroids (SOSRS)/brain organoids (SOSRS) comprising neural progenitors and neurons having a ventral cell fate from pluripotent stem cells tissue.

18. The method of claim 17, wherein the pluripotent stem cells are a two-dimensional monolayer of pluripotent stem cells.

19. The method of claim 17,
wherein inhibiting the BMP signaling pathway comprises culturing the pluripotent stem cells with a small molecule that inhibits the BMP signaling pathway,
wherein inhibiting the Wnt signaling pathway comprises culturing the pluripotent stem cells with a small molecule that inhibits the Wnt signaling pathway,
wherein inhibiting the TGFβ signaling pathway comprises culturing the pluripotent stem cells with a small molecule that inhibits the TGFβ signaling pathway,
wherein activating the SHH signaling pathway comprises culturing the pluripotent stem cells with a small molecule that activates the SHH signaling pathway.

20. The method of claim 19,
wherein the small molecule that inhibits the BMP signaling pathway is selected from the group consisting of 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyri-midine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347),
wherein the small molecule that inhibits the Wnt signaling pathway is selected from the group consisting of N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phe-nylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thiol-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyri-midin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein 1 (SFRP1),
wherein the small molecule that inhibits the TGFβ signaling pathway is selected from the group consisting of 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinox-aline (SB525334), 2-(5-Benzo[1,3]dioxol-5-yl-2-ieri-butyl-3H-imidazol-4-yl)-6-methylpyridin-e hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-be-nzamide hydrate, left-right determination factor (Lefty), 3-(6-Methyl-2-pyridinyl)-N-phenyl-4-(4-quinolinyl)-1H-pyrazole-1-carbothi-oamide (A 83-01), 4-[4-(2,3-Dihydro-1,4-benzodioxin-6-yl)-5-(2-pyridinyl)-1H-imidazol-2-yl]-benzamide (D 4476), 4-[4-[3-(2-Pyridinyl)-1H-pyrazol-4-yl]-2-pyridinyl]-N-(tetrahydro-2H-pyra-n-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazo-le-1-ethanol (R 268712) or 2-(3-(6-Methylpyridine-2-yl)-1H-pyrazol-4-yl)-1,5-naphthyridine (RepSox),
wherein the small molecule that activates the SHH signaling pathway is smoothened agonist (SAG).

21-26. (canceled)

27. The method of claim 17, wherein the culturing is for approximately four days.

28. The method of claim 17, further comprising transferring a portion of the cultured pluripotent stem cells onto a gelatinous basement membrane matrix.

29. (canceled)

30. The method of claim 17, wherein the SOSRS express markers for of ventral forebrain, wherein the markers for of ventral forebrain are NKX2.1, GABA, and somatostatin.

31. (canceled)

32. The method of claim 17, wherein the SOSRS generate GABAergic interneurons.

33. The method of claim 1, wherein the culturing steps are conducted in vitro.

34. A composition comprising self-organizing single-rosette spheroids (SOSRS)/brain organoids comprising neural progenitors and neurons having a dorsal cell fate or a ventral cell fate.

35-55. (canceled)

56. The method of claim 17, wherein the culturing steps are conducted in vitro.
